(19) United States
(12) Patent Application Publication
(10) Pub. No.: US 2010/0198196 A1
(43) Pub. Date: Aug. 5, 2010

(54) Therapy Delivery Device Programming Tool

(75) Inventor: Charles Wei, Fremont, CA (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 12/363,715

(22) Filed: Jan. 30, 2009

(51) Int. Cl. A61M 5/172

(52) U.S. Cl. 604/890.1

(57) ABSTRACT

Methods and devices for providing programming tool for programming the initial and on-going operation of a therapy delivery device is provided. Embodiments include generating empirical rules for determining initial operating settings, applying the final empirical rules to a patient population, detecting a connection to a therapy delivery device, identifying the connected delivery device, receiving data download request from the delivery device. retrieving updated data associated with the connected delivery device; visualizing changes to the settings data of the device, and automatically transmitting the retrieved updated data to the delivery device.

### BACKGROUND

Diabetic patients typically administer insulin to sustain their physiological conditions. Typically, these patients administer doses of either fast acting or slow acting insulin using needle type syringes, for example, prior to meals, and/or at a suitable time during the course of each day contemporaneously with the blood glucose level testing using fingerstick blood glucose testing, for example. If insulin is not suitably administered, the diabetic patients risk serious if not fatal damage to the body.

Continued development and improvement in the external infusion pump therapy in recent years have drawn much appeal to the diabetic patients for, among others, improved management of diabetes by better regulating and controlling the intake of insulin. Typically, the patient inserts a cannula which is connected to as infusion tubing attached to an external pump, and insulin is administered based on a preprogrammed basal profiles. Moreover, the external infusion devices presently available include computational capability to determined suitable bolus doses such as carbohydrate bolus and correction bolus, for example, to be administered in conjunction with the infusion device executing the patient's basal profile.

Commercially available infusion devices such as insulin pumps are programmable and include sophisticated functionalities to enhance therapy management. Consistent with the sophistication of these devices, it is often cumbersome and trying to program these complex devices to the desired delivery, notification, modification and/or other adjustments or settings to be suitable for each user's physiology or therapy. Often, the infusion devices are miniaturized to provide each of portability and comfort in extended wear or use. However, with miniaturization necessarily follows compact or miniaturized user interface including, for example, small display screens, compact buttons or input mechanisms and the like.

Initial programming of such compact devices, for example, may be challenging and include a steep learning curve, often times dissuading the user from the use of the devices themselves, or modifying the existing programming in the infusion device.

### SUMMARY

Embodiments of the subject disclosure include methods device for programming a therapy delivery device such as a medication infusion device including, for example insulin pump. More particularly, embodiments of the present disclosure include generating and customizing empirical rules for deriving initial delivery settings, automating the application of these rules for patient use, detecting a connection to a therapy delivery device, identifying the connected delivery device, receiving data download request from the delivery device, retrieving updated data associated with the connected delivery device, allowing user to review and confirm the changes, and automatically transmitting the retrieved updated data to the delivery device.

Also provided are systems, computer program products, and kits.

### BRIEF DESCRIPTION OF THE DRAWINGS

FIG. 1 shows a block diagram of an overall therapy delivery device programming tool in accordance with one aspect;

FIG. 2 is a flowchart illustrating a routine for programming a therapy delivery device in accordance with one aspect of the present disclosure;

FIG. 3 is a flowchart illustrating a routine for programming a therapy delivery device in accordance with another aspect of the present disclosure; and

FIGS. 4-21 are screen shot illustrations of the overall therapy delivery device programming tool in accordance with one aspect of the present disclosure.

### DETAILED DESCRIPTION

Within the scope of the present disclosure, there are provided method and system for programming a therapy delivery device such as an infusion device via a remote terminal automatically, or semi-automatically, where the therapy profile or prescription is entered via the remote terminal such as a computer terminal and thereafter transferred to the therapy delivery device upon establishing communication link between the device and the remote terminal. In aspects of the present disclosure, the user or the healthcare provider may, enter the appropriate therapy profile in the remote terminal via its user interface, and upon establishing a communication link with the therapy delivery device, the therapy profile is uploaded into the therapy delivery device automatically or upon acknowledgement or confirmation by the user, to program the therapy delivery device and thereafter for execution of the therapy profile.

In one embodiment, using the remote terminal, the physician or the user or patient of the therapy delivery device may program one or more suitable basal delivery profiles, alarm or alert notification parameters, and other appropriate therapy or device related parameters, and when a communication link is established between the device and the remote terminal, the programmed basal delivery profiles, alarm or alert notification parameters and/or other appropriate therapy or delivery device related parameters are transferred to the device for execution and/or storage.

In one aspect, there is provided an automated approach to process existing empirical rules or relationships used by the healthcare provides to determine the initial insulin delivery settings.

In one embodiment, the empirical rules or relationships may be integrated into the delivery device configuration program, minimizing potential error in the translation of the settings, parameters, and/or configurations.

Accordingly, in one embodiment, healthcare providers may be provided with an automated tool to extract the relevant information from the patient or the user and to program the appropriate parameters to the delivery device.

In one embodiment, the empirical rules may be generalized and customized for different healthcare providers, (each may use different rules depending on how conservative they are at the initial setup, and the patient type and levels of control). They can apply their own set of rules to their patients. In one aspect, having an automated tool that removes the paper transcription and translation process may reduce errors and improve workflow.

Before the present disclosure is described in additional detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

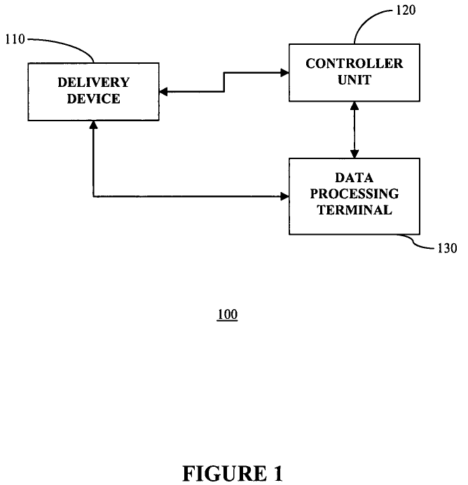

FIG. 1 shows a block diagram of an overall therapy delivery device programming tool in accordance with one aspect of the present disclosure. Referring to the Figure, the system for providing therapy delivery device programming tool 100 includes a therapy delivery device 110 capable of data communication with a controller unit 120 and a data processing terminal 130. The therapy delivery device 110 in one embodiment may include an external infusion device such as an external insulin pump, an implantable infusion device, an on-body micropump device, a transdermal delivery device, or any other suitable microprocessor controlled medication delivery device.

In one aspect, the therapy delivery device 110 may include one or more user interface components such as a display, input buttons, speakers for providing audible or audio output, and the like. In one aspect, the therapy delivery device is ambulatory and configured to be carried around by the user. In still a further aspect, the therapy delivery device 110 may include a communication component which allows the device 110 to connect to or communicate with one or more of the controller unit 120 and the data processing terminal 130. The communication link between the therapy delivery device 110 and the one or more of the controller unit 120 and the data processing terminal 130 may include RF communication, Infrared communication, 801.x1 based communication protocol, cellular network based communication protocol and the like. In accordance with aspects of the present disclosure, the communication between the various components of the overall system 100 shown in FIG. 1 may also includes wires connection using, for example, USB connection, RS-232 connection, or any other suitable data transfer connection.

In one aspect, the therapy delivery device 110 may be configured to deliver medication such as insulin using an infusion set connectable to the housing of the device and in fluid communication with one or more reservoirs housed within the device 110, where the infusion set includes a cannula for medication delivery to the user or patient. In a further aspect, the therapy delivery device 110 is microprocessor driven and controlled by one or more microprocessor units to execute functions related to the medication delivery, operational condition monitoring of the therapy delivery device, and the like.

Referring back to FIG. 1, also shown is the controller unit 120 which may include a wireless or wired communication device which is configured to control at least in part, the operation of the therapy delivery device, and further, configured to communicate with the data processing terminal 130. Each communication link between the various components shown in FIG. 1 including the therapy delivery device 110, the controller unit 120, and the data processing terminal 130 may be bi-directional. In one aspect, the controller unit 120 may include some or all of the functionalities provided in the therapy delivery device 110 including the control of the medication dispensing protocol, condition notification such as detected occlusion in the infusion tubing, and/or along the fluid path from the therapy delivery device 110 to the user, programming, controlling or monitoring the operation of the therapy delivery device 110.

Optionally, the controller unit 120 may include a strip port for receiving an in vitro blood glucose test strip for determining a blood glucose measurement. Additionally, the controller unit 120 may include the functionalities of an analyte monitoring device which may be configured to be in communication with an analyte sensor for monitoring an analyte level of a user. Further detailed descriptions of embodiments of the continuous analyte monitoring system, embodiments of its various components are provided in U.S. Pat. No. 6,175,752 issued Jan. 16, 2001 entitled “Analyte Monitoring Device and Methods of Use”, and in application Ser. No. 10/745,878 filed Dec. 26, 2003 entitled “Continuous Glucose Monitoring System and Methods of Use”, each assigned to the Assignee of the present application, disclosure of each of which are incorporated by reference for all purposes.

Referring back to FIG. 1, the data processing terminal 130 in one aspect may include a personal computer, a portable computer such as a laptop or a handheld device (e.g., personal digital assistants (PDAs), telephone such as a cellular phone (e.g., a multimedia and Internet-enabled mobile phone such as an iPhone or a Blackberry device), mp3 player, pager, and the like), each of which may be configured for data communication with the delivery device 110 and/or the controller unit 120 via wired or wireless connection. Additionally, the data processing terminal 130 may further be connected to a data network (not shown) for storing, retrieving, updating, and/or analyzing data associated with the therapy delivery device 110 as well as the controller unit 120.

In this manner, in accordance with aspects of the present disclosure, programming or modification of parameters and/or operational conditions for the therapy delivery device 110 may be directly input to the data processing terminal 130 which may include a more user friendly user interface (for example, a larger display, full size keyboard or other input device, as well as software programming which allows for the entry of the appropriate operational parameters or functions associated with the delivery device 120 such as a software configured to be responsive to the user input values or parameters for the operation of the delivery device 110.

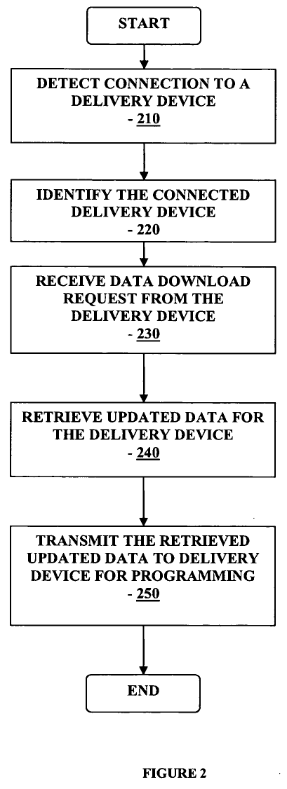

FIG. 2 is a flowchart illustrating a routine for programming a therapy delivery device in accordance with one aspect of the present disclosure. Referring to the Figure, initially a communication link or commenction to the delivery device 110 (FIG. 1) is detected by the data processing terminal (210). Thereafter, the identity of the connected delivery device 110 (FIG. 1) is determined (220), for example, based on the programmed or stored user name, device serial number, or a unique user account number associated with the delivery device connected to the terminal (120). Within the scope of the present disclosure, other techniques for determining the identity of the connected delivery device may be used, such as, for example, but not limited, based on encryption/decryption keys, respective unique device/terminal serial numbers, pre-stored communication protocol information, and the like.

Referring back to FIG. 2, after identifying the connected delivery device, data download request from the delivery device may be received (230). For example, the therapy delivery device may be programmed to transmit a request to upload its stored data to synchronize with the connected data processing terminal 130 (FIG. 1). In one aspect, the data download request may be in response to the successful establishment of the communication link and/or proper identification of the respective connected devices. In another aspect, the data processing terminal 130 may be configured to generate a request to upload the data from the deliver device 110. Referring again to FIG. 2, the data processing terminal 130 may be configured to, after receiving the data or parameters from the delivery device, synchronize the data, reconciling any data conflicts including discrepancies in parameter or stored information, and thereafter, retrieve updated data to transmit to the therapy delivery device for execution and storage therein (240).

For example, in one aspect, the user or the physician may have established or stored in the data processing terminal 130 suitable modification to the therapy profile, one or more new therapy profiles, modified physiological and/or device related notification parameters such as alarms, and/or alerts, prior to the connection with the therapy delivery device, which may then be transferred to the delivery device for storage and/or execution (250).

In the manner described, in accordance with embodiments of the present disclosure, therapy delivery device 110 (FIG. 1) programming or control may be implemented via a data processing terminal 130 connected to the therapy delivery device 110. The data processing terminal 130 may include software or tools that enable the user to assess different parameters related to the operation of the delivery device 110 and/or the underlying therapy itself. The delivery device programming may be achieved readily and easily using the data processing terminal 130 in one or more operations executed at the data processing terminal 130.

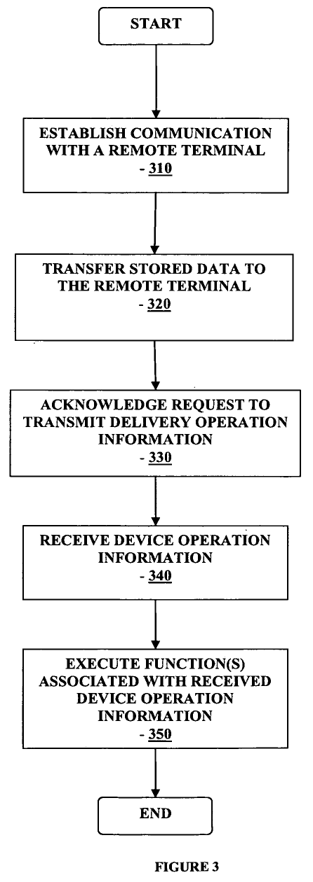

FIG. 3 is a flowchart illustrating a routine for programming a therapy delivery device in accordance with another aspect of the present disclosure. Referring to FIG. 3, in one aspect of the present disclosure, communication with a remote terminal such as the controller unit 120 and/or the data processing terminal 130 may be established by the delivery device 310. That is, the communication may be initiated from the delivery device 310 and established based on one or more predetermined communication protocol. Thereafter, stored data from the delivery device 110 (FIG. 1) is transferred to the remote terminal to be synchronized. In one aspect, this communication is a unidirectional communication from the delivery device to the remote terminal.

In another aspect, this communication may be bi-directional where the stored data transfer may be implemented in both directions either simultaneously or sequentially, and further, where each device (therapy device and the remote terminal) includes programming to resolve any data, information or parameter conflicts detected. In this manner, once the communication link between the delivery device and the remote terminal is established, each device or one or more devices within the overall system 100 (FIG. 1) may include programming to reconcile any discrepancies between the data from the two sources.

Referring back to FIG. 3, in one aspect, the delivery device 110 (FIG. 1) may be configured to acknowledge request to transmit delivery device operation information received from the remote terminal (330). Thereafter, the operation information may be received from the remote terminal or any device in signal communication that was the source of the transmitted request (340). The therapy delivery device in one aspect may be configured to store the received device operation information, and further, may be configured to execute one or more function associated with the received device operation information (350).

In the manner described, in accordance with aspects of the present disclosure, programming of the therapy delivery device 110 (FIG. 1) may be remotely performed via a remote terminal such the data processing terminal 130 and/or the controller unit 120, where one or more preprogrammed or prestored therapy delivery profile and/or operational control parameters are transferred to the delivery device automatically or in response to a transmit request from the delivery device 110. Alternatively, the delivery device 110, while connected to the data processing terminal 130, may be programmed in real time, based on the data or information input to the data processing terminal 130 which is substantially contemporaneously transferred to the delivery device 110.

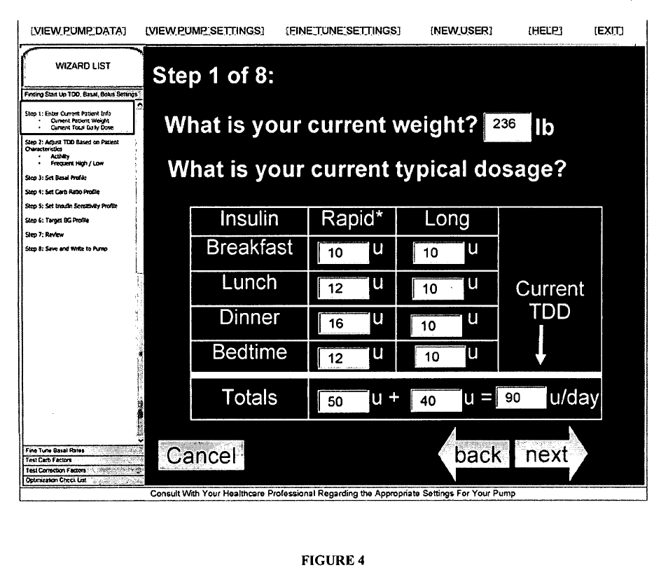

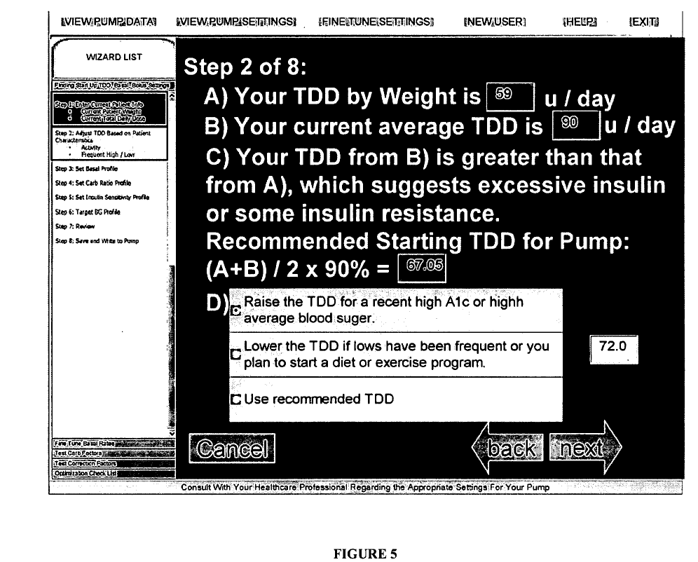

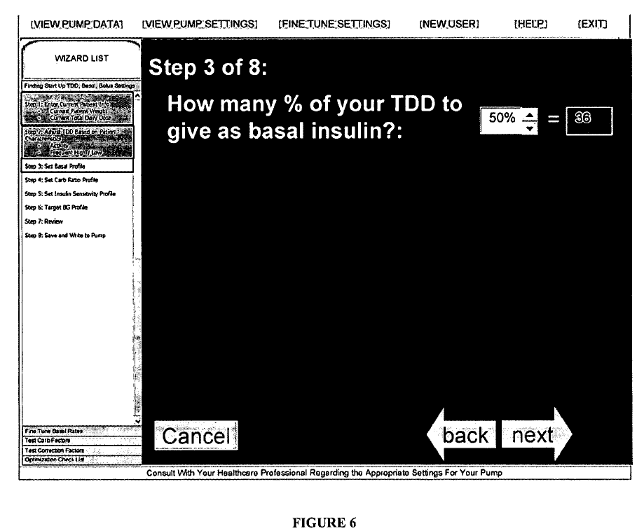

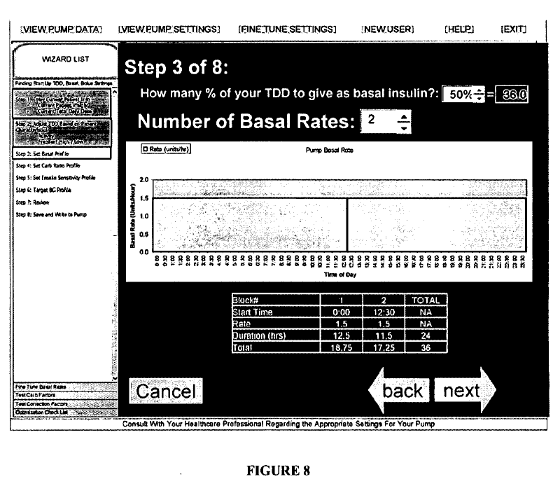

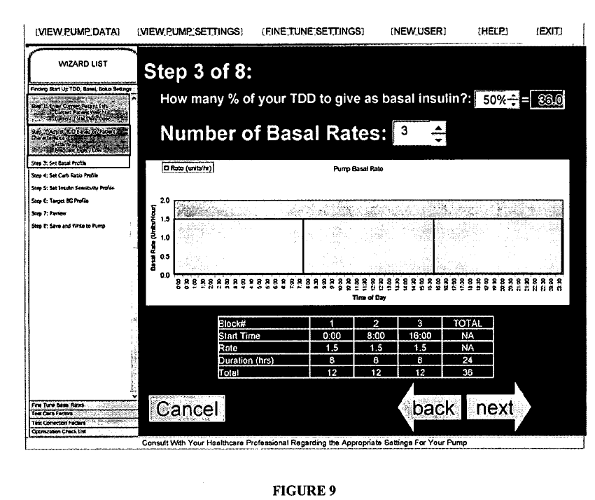

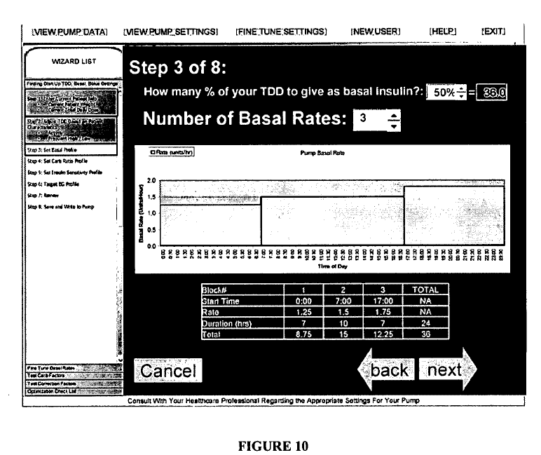

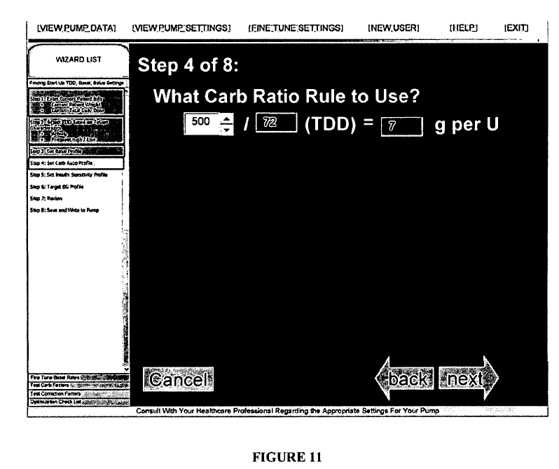

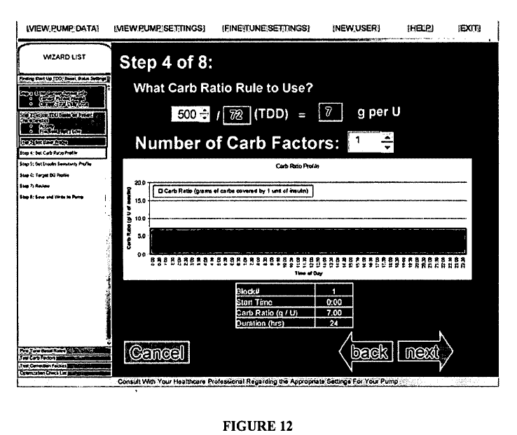

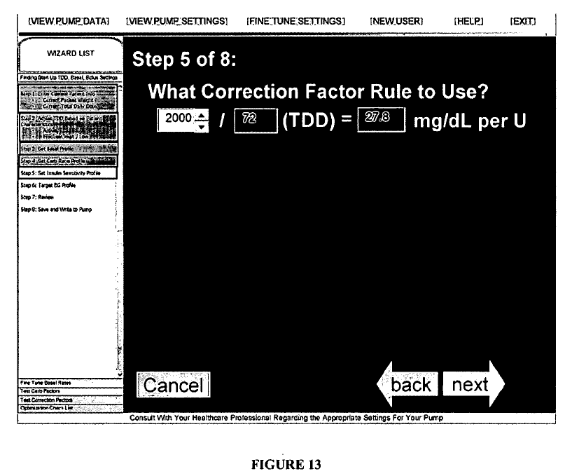

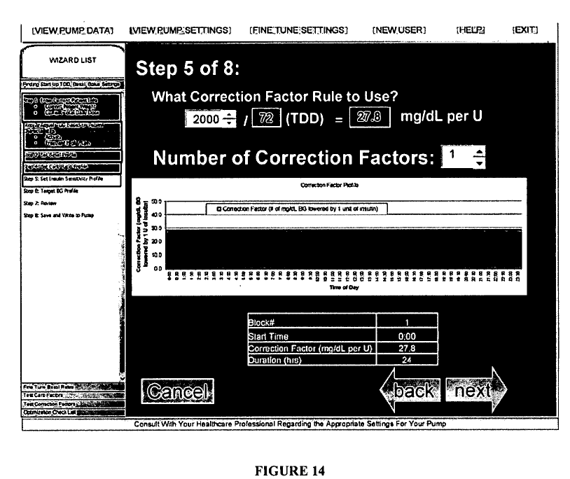

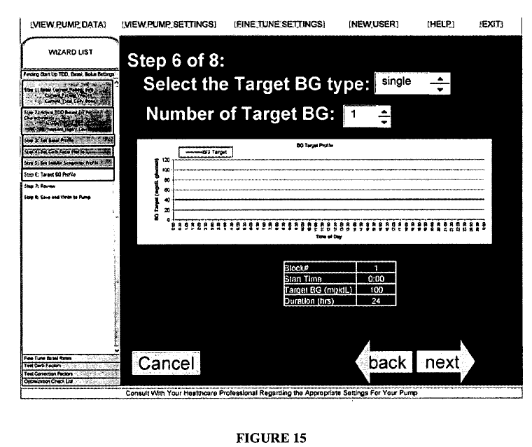

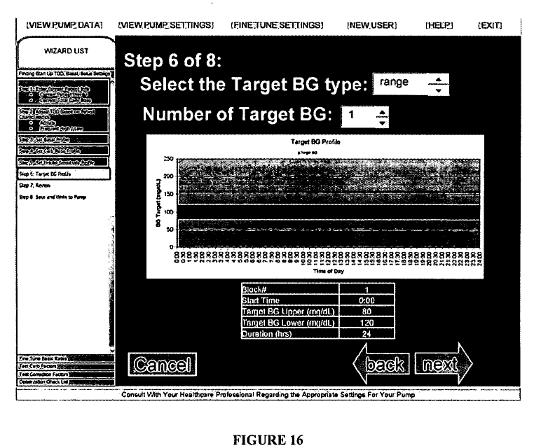

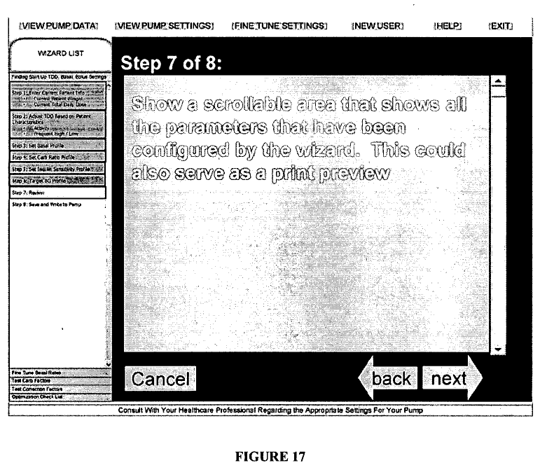

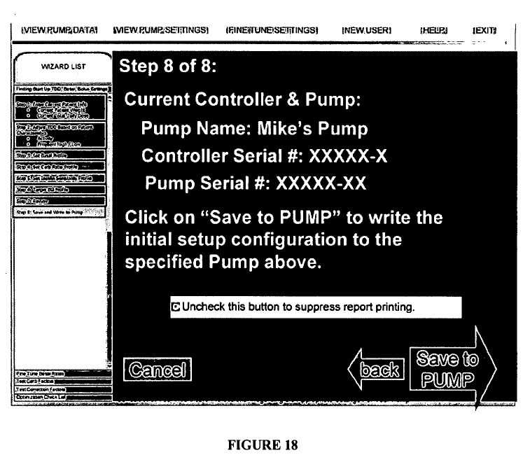

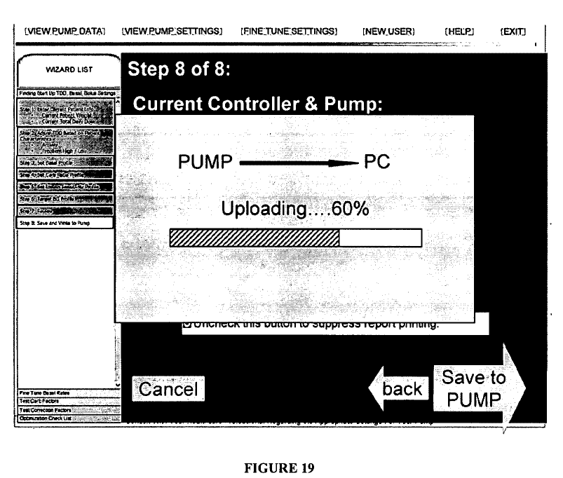

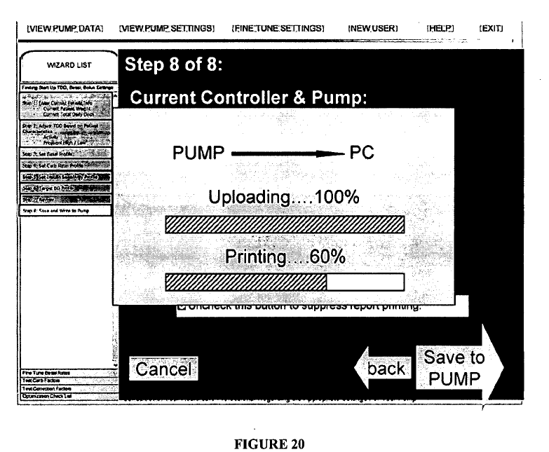

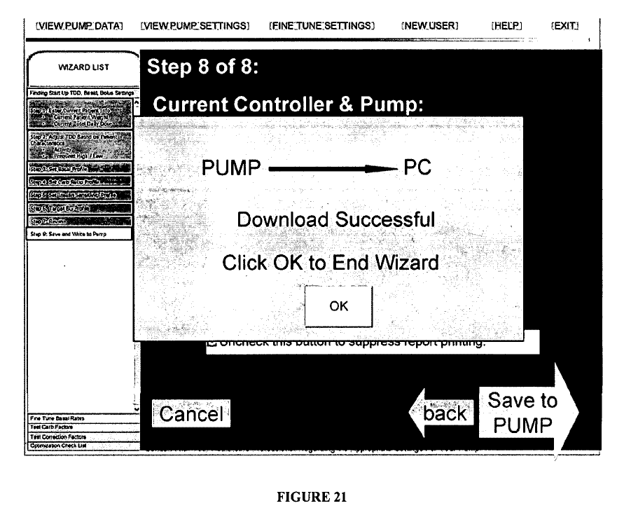

FIGS. 4-21 are screen shot illustrations of the overall therapy delivery device programming tool in accordance with one aspect of the present disclosure. As can be seen, by way of exemplary screen displays, for example, shown on the user interface of the data processing terminal 130 (FIG. 1) for example, each step or routine associated with the programming of an initial settings on a insulin pump is shown. For example, FIG. 4 shows a screen shot illustrating user input of the weight information as well as current typical insulin dosage information based on meal events and evening/bedtime. FIG. 5 illustrates additional parameters or variables that may be entered and which are relevant to the therapy profile.

In addition to the user inputted information, as shown in FIG. 5, the programming tool resident and operating in the data processing terminal 130 (FIG. 1) may be configured to perform calculations or processings based at least in part, on the information provided by the user. In one aspect, the patient or the users are provided tools (or user interface components, for example) to allow them to generate and/or customize the rules. The rules may vary or differ depending upon the treating physician and/or the patient or user. The user may be also provided with a list of questions and answers that they enter into the data processing terminal 130, for example. Furthermore, formulas or relationships as to how the various inputted parameters or variables are provided to generate the corresponding or appropriate initial basal delivery profile, insulin sensitivity information, carbohydrate ratio and the like for the initial delivery device start up procedure. In one aspect, the initial delivery device start up procedure and/or the parameters may be configured to be relatively conservative. In one aspect, after the initial delivery device profile setup, the user information may be entered or provided to apply these rules or relationships to the specific user.

In another aspect, each of these steps and the order of the steps may be modified by the physician based on their preferences. For example, the empirical number to generate the carbohydrate to insulin ratio and insulin correction factor may be adjusted based on the level of control of the patient, the knowledge of the patient, and how conservative the physician likes to be getting a new user on the pump.

Also, the physician may add a whole new step to query a particular relevant health state of the patient which may be associated with a more aggressive or more conservative approach to the initial insulin delivery setting. Some relevant parameters or queries may include frequency of hypoglycemic or hyperglycemic events, frequency of exercise and its intensity, and the like. Once configured, the series of steps can be used to improve the workflow of setting up initial settings for additional patients.

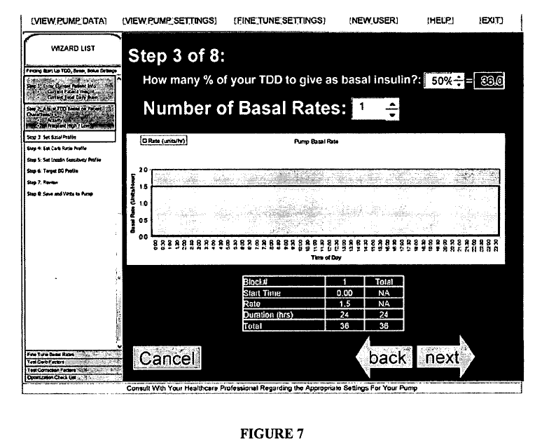

Also shown in these Figures are graphical illustrations associated with the programming tool, where the dosage information may by visually presented and using the user interface, such as a computer mouse, the dosing parameters may be modified, for example, by selecting and moving the cursor relative to the graphical display shown in for example, FIGS. 7-10.

Indeed, as shown in FIGS. 4-21, the data processing terminal 130 in one embodiment may be provided with a programming tool which receives patient specific data, and in addition perform calculations and/or determination to provide the initial startup therapy treatment regimen which is then communicated to the delivery device 110 for execution.

In this manner, in accordance with one aspect of the present disclosure, there is provided formulating one or more known or customized empirical rules to determine the relevant patient information for deriving at initial delivery device operational settings, applying the known or customized empirical rules, automatically transferring the initial delivery device configuration settings to a delivery device, where the automatically transferring may include detecting a connection to a therapy delivery device, identifying the connected delivery device, receiving or initiating data download request from the delivery device, retrieving updated data associated with the connected delivery device, reviewing and confirming the changes, and automatically transmitting the retrieved updated data to the delivery device.

One aspect may include formulating one or more of generating new queries, modifying the order of query presentation, or utilizing one or more new empirical values to determine one or more of an insulin sensitivity or a carbohydrate to insulin ratio.

Identifying the connected delivery device may include comparing an identification information received from the delivery device to a prestored information associated with the delivery device.

The retrieved updated data may include programming data for the therapy delivery device.

The programming for the therapy delivery device may include fluid dispensing protocol.

The fluid dispensing protocol may include one or more of a basal delivery profile, or a bolus delivery profile.

The retrieved updated data may include one or more operational parameters associated with the operation of the delivery device.

One aspect may include synchronizing the data from the delivery device with stored data.

Another aspect may include reconciling the data from the delivery device with stored data.

In another embodiment, a method may comprise, establishing communication with a remote terminal, transferring stored data to the remote terminal, acknowledging request to transmit delivery operation information, receiving device operation information, visualizing changes in the device operation information for comparison and confirmation, and executing one or more functions associated with the received device operation information.

One aspect may include automatically implementing the device operation information after disconnecting with the remote terminal or the pump.

Another aspect may include displaying the current parameter residing on the delivery device and the new parameter to be sent to the device and executed on the delivery device.

In another embodiment, an apparatus may comprise, a data communication interface, one or more processors operatively coupled to the data communication interface, and a memory for storing instructions which, when executed by the one or more processors, causes the one or more processors to detect a connection to a therapy delivery device, identify the connected delivery device, receiving data download request from the delivery device, to retrieve updated data associated with the connected delivery device, and automatically transmit the retrieved updated data to the delivery device.

The memory for storing instructions which, when executed by the one or more processors, may cause the one or more processors to compare an identification information received from the delivery device to a prestored information associated with the delivery device.

The retrieved updated data may include programming data for the therapy delivery device.

The memory for storing instructions which, when executed by the one or more processors, may cause the one or more processors to program for the therapy delivery device including fluid dispensing protocol.

The fluid dispensing protocol may include one or more of a basal delivery profile, or a bolus delivery profile.

The retrieved updated data may include one or more operational parameters associated with the operation of the delivery device.

The memory for storing instructions which, when executed by the one or more processors, may cause the one or more processors reconciling the data from the delivery device with stored data.

In another embodiment of the disclosure, one or more storage devices having processor readable code embodied thereon, said processor readable code for programming one or more processors to estimate an analyte level may comprise, detecting a connection to a therapy delivery device, identifying the connected delivery device, receiving data download request from the delivery device, retrieving updated data associated with the connected delivery device, and automatically transmitting the retrieved updated data to the delivery device.

The various processes described above including the processes performed by the delivery device 110, the controller unit 120, and the data processing terminal 130 (FIG. 1) in the software application execution environment in the overall system for providing delivery device programming tool 100 including the processes and routines described in conjunction with FIGS. 2-3, may be embodied as computer programs developed using an object oriented language that allows the modeling of complex systems with modular objects to create abstractions that are representative of real world, physical objects and their interrelationships. The software required to carry out the inventive process, which may be stored in the memory or storage device (not shown) of the delivery device 110, the controller unit 120, and the data processing terminal 130, may be developed by a person of ordinary skill in the art and may include one or more computer program products.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

What is claimed is:

1. A method, comprising:
formulating one or more known or customized empirical rules to determine the relevant patient information for deriving at initial delivery device operational settings;
applying the known or customized empirical rules;
automatically transferring the initial delivery device configuration settings to a delivery device, the automatically transferring including:
detecting a connection to a therapy delivery device;
identifying the connected delivery device;
receiving or initiating data download request from the delivery device;
retrieving updated data associated with the connected delivery device;
reviewing and confirming the changes; and
automatically transmitting the retrieved updated data to the delivery device.

2. The method of claim 1 wherein formulating includes formulating one or more of generating new queries, modifying the order of query presentation, or utilizing one or more new empirical values to determine one or more of an insulin sensitivity or a carbohydrate to insulin ratio.

3. The method of claim 1 wherein identifying the connected delivery device includes comparing an identification information received from the delivery device to a prestored information associated with the delivery device.

4. The method of claim 1, wherein the retrieved updated data includes programming data for the therapy delivery device.

5. The method of claim 4 wherein the programming for the therapy delivery device includes fluid dispensing protocol.

6. The method of claim 5 wherein fluid dispensing protocol includes one or more of a basal delivery profile, or a bolus delivery profile.

7. The method of claim 1 wherein the retrieved updated data includes one or more operational parameters associated with the operation of the delivery device.

8. The method of claim 1 including synchronizing the data from the delivery device with stored data.

9. The method of claim 1 including reconciling the data from the delivery device with stored data.

10. A method, comprising:
establishing communication with a remote terminal;
transferring stored data to the remote terminal;
acknowledging request to transmit delivery operation information;
receiving device operation information;
visualizing changes in the device operation information for comparison and confirmation; and
executing one or more functions associated with the received device operation information.

11. The method of claim 10 including automatically implementing the device operation information after disconnecting with the remote terminal or the pump.

12. The method in claim 10 including displaying the current parameter residing on the delivery device and the new parameter to be sent to the device and executed on the delivery device.

13. An apparatus, comprising:
a data communication interface;
one or more processors operatively coupled to the data communication interface; and
a memory for storing instructions which, when executed by the one or more processors, causes the one or more processors to detect a connection to a therapy delivery device, identify the connected delivery device, receiving data download request from the delivery device, to retrieve updated data associated with the connected delivery device, and automatically transmit the retrieved updated data to the delivery device.

14. The apparatus of claim 13 wherein the memory for storing instructions which, when executed by the one or more processors, causes the one or more processors to compare an identification information received from the delivery device to a prestored information associated with the delivery device.

15. The apparatus of claim 13 wherein the retrieved updated data includes programming data for the therapy delivery device.

16. The apparatus of claim 15 wherein the memory for storing instructions which, when executed by the one or more processors, causes the one or more processors to program for the therapy delivery device including fluid dispensing protocol.

17. The apparatus of claim 16 wherein fluid dispensing protocol includes one or more of a basal delivery profile, or a bolus delivery profile.

18. The apparatus of claim 13 wherein the retrieved updated data includes one or more operational parameters associated with the operation of the delivery device.

19. The apparatus of claim 13 including synchronizing the data from the delivery device with stored data.

20. The apparatus of claim 13 wherein the memory for storing instructions which, when executed by the one or more processors, causes the one or more processors reconciling the data from the delivery device with stored data.

21. One or more storage devices having processor readable code embodied thereon, said processor readable code for programming one or more processors to estimate an analyte level, comprising:
detecting a connection to a therapy delivery device;
identifying the connected delivery device;
receiving data download request from the delivery device;
retrieving updated data associated with the connected delivery device; and
automatically transmitting the retrieved updated data to the delivery device.
